(19) United States
(12) Patent Application Publication
(10) Pub. No.: US 2011/0308240 A1
(43) Pub. Date: Dec. 22, 2011

(54) DEGRADATION OF BIOMASS BY METAL-CATALYZED PEROXIDE OXIDATION

(75) Inventors: Ali Zendedel Haghighi, San Jose, CA (US); Javad J. Sahbari, Santa Rosa, CA (US)

(73) Assignee: Applied Chemical Laboratories, Inc., Sunnyvale, CA (US)

(21) Appl. No.: 13/162,478

(22) Filed: Jun. 16, 2011

Related U.S. Application Data
(60) Provisional application No. 61/355,392, filed on Jun. 16, 2010.

(51) Int. Cl. F15B 13/00; C12P 19/02; F27B 17/00; C10G 1/00; C01B 3/02; C07H 1/08; C10J 3/46

(52) U.S. Cl. 60/325; 536/128; 435/105; 48/197 R; 585/240; 423/648.1; 432/1

(57) ABSTRACT

This invention relates to degradation of biomass with a peroxide in the presence of a metal catalyst. The process can result in ethanol production from cellulosic biomass, fuel gases, useful chemicals and biochemicals from decomposition products, heat and/or pressure for direct use, and electricity generation.

### RELATED APPLICATIONS

This application claims the benefit of U.S. Provisional Patent Application Ser. No. 61/355,392 filed Jun. 16, 2010.

### FIELD OF THE INVENTION

This invention relates to degradation of biomass with a peroxide in the presence of a metal catalyst.

### BACKGROUND OF THE INVENTION

Ethanol has been used as an alternative or additive form of fuel. It has been used either as an alternative fuel to gasoline or as an additive to gasoline. About 70% of the ethanol is produced through fermentation. In this process, carbohydrates (mainly glucose) are added to a mixture containing a bacteria or yeast, where due to the fermentation, ethanol is produced. The main source of feedstock for the fermentation process is corn, wheat or soybean. However, due to the fact that these feedstocks are also main sources of food, use of these carbohydrate resources for fuel has become problematic. Hence, an alternative has been proposed, where cellulosic biomass feedstock are used. Ethanol generated by this means is called cellulosic ethanol. Shell Oil has predicted “the global market for biofuel such as cellulosic ethanol will grow to exceed $10 billion by 2012”. In this process, cellulosic biomass, which includes agricultural wastes (such as cereal straws), plant wastes from industrial processes (such as sawdust and paper pulp), and municipal solid waste, can be used. However, in the case of cellulosic biomass the sugars needed for the fermentation are locked in the polysaccharides, so the separation of these complex polymeric structures into fermentable sugars is essential to the efficient and economical production of cellulosic ethanol. There are two options employed for this purpose. One option utilizes acid hydrolysis to break down the complex carbohydrates into simple sugars. The other option uses enzymatic hydrolysis, to obtain the simple sugars. In either option, the cellulosic biomass needs to be broken up in order to get the simple sugars. This is performed by either mechanical and/or thermal means, both of which are costly.

It has been reported that oxidation of cellulose, results in the degradation of the polymer. Illman, et.al., have shown that the depolymerization of cellulose in brown-rot fungi colonized wood, is due to the oxidative damage (Biodeterioation Research 2, 1989, 497-509). Hyde et. al., have shown that the hydroxyl radicals are the causative agents (Microbiology, 1997, 143, 259).

Hydrogen peroxide can decompose to water and oxygen, in the presence or absence of metal cations. Rate of decomposition is much higher in the presence of metal cation catalysts. This decomposition, in the presence of metal cations, such as iron or copper, occurs through a mechanism called the Fenton reaction. Oxygen free radicals are produced during the Fenton reaction. Hence, the cellulosic material in biomass can be degraded in the presence of hydrogen peroxide and a metal cation catalyst such as copper.

### SUMMARY OF THE INVENTION

Biomass, either made onsite (i.e. a mixture of grass and banana peels), or obtained from industry (i.e. switch grass, sawdust, hog fuel or corn stover), was treated with hydrogen peroxide at different concentrations, in the presence or absence of copper foil or wire. Degradation of the biomass was calculated by weighing the biomass before and after the treatment. The result was expressed in term of percent degradation. As a control, the onsite produced biomass was treated with 40% sulfuric acid. Benedict's test, glucose oxidase assay, HPLC and MS methods were used to detect the presence of glucose. A refractometer was used to measure the relative amount of glucose and hydrogen peroxide present in solutions.

A small incubator has been built as a prototype and the temperature and pressure measurements of the oxidized glucose in the presence of hydrogen peroxide and copper wire has been made.

### BRIEF DESCRIPTION OF THE DRAWINGS

FIG. 1 shows degradation of biomass with different hydrogen peroxide concentrations in the presence of copper.

FIG. 2 illustrates degradation of switch grass, sawdust, hog fuel, and corn stover with peroxide in the presence of copper.

FIG. 3 shows HPLC results for glucose production from different types of biomass.

FIG. 4 shows mass spectrometer confirmation of glucose in decomposition of biomass:

FIG. 5 shows heat generated from a mixture of glucose, copper, and hydrogen peroxide.

FIG. 6 shows acceleration of heat generation in biomass decomposition with peroxide/copper when UV light exposure is used.

FIG. 7 illustrates heat generation during a reaction.

FIG. 8 shows the inverse correlation between copper hydroxide concentration and the time for a reaction mixture to reach boiling temperature.

### DETAILED DESCRIPTION

Onsite made biomass (8 grams), was treated with either 50 ml hydrogen peroxide (30%) and copper foil (0.2 g), or 50 ml sulfuric acid (40%). The combination of hydrogen peroxide and copper could completely degrade the biomass, while 40% sulfuric acid could not accomplish the same.

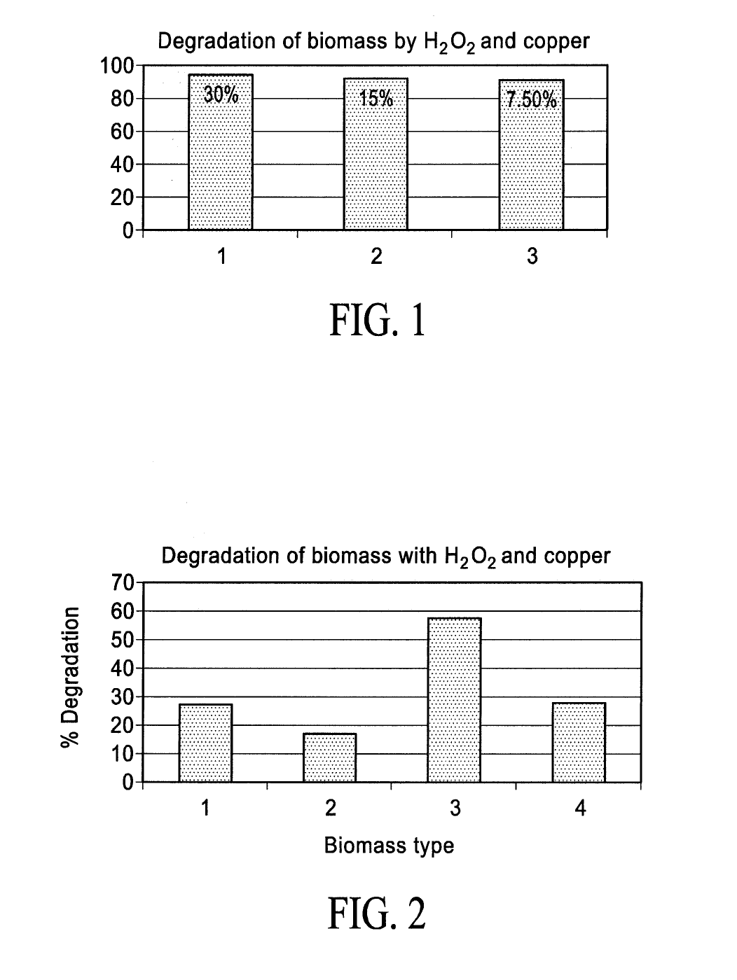

Next, biomass (2 g) was treated with different concentrations of hydrogen peroxide in the presence of copper (0.2 g). As shown in the FIG. 1, more than 90% degradation was obtained even at a hydrogen peroxide concentration of 7.5%.

To demonstrate the role of the metal catalyst in the degradation, the biomass was treated in the presence of hydrogen peroxide (30%), with or without copper. Biomass treated with copper and distilled water was used as the control. Biomass remained intact in the presence of distilled water and copper or hydrogen peroxide alone, but it was degraded in the presence of hydrogen peroxide and copper.

Next, the degradation effect of hydrogen peroxide and copper was demonstrated by using the types of biomass used by industry. Four different types of biomass were used. Switch grass (#1), sawdust (#2), hog fuel (#3) and corn stover (#4). The different biomass was treated in the presence of hydrogen peroxide (30%) and copper wire. As described above, percent degradation was calculated by weighing the biomass before and after the treatment. As shown in the FIG. 2, all four types of biomass could be degraded by the treatment.

To demonstrate the effectiveness of treatment in the extraction of glucose from the biomass and the role of catalysts in this process, following experiments were performed:

Sawdust (2.0 g) treated with hydrogen peroxide at 35% and copper wire (0.1 g) in a total volume of 30 ml. The resulting liquid was treated with the Benedict's test. A red precipitate is a positive control for reducing surges, which includes glucose.

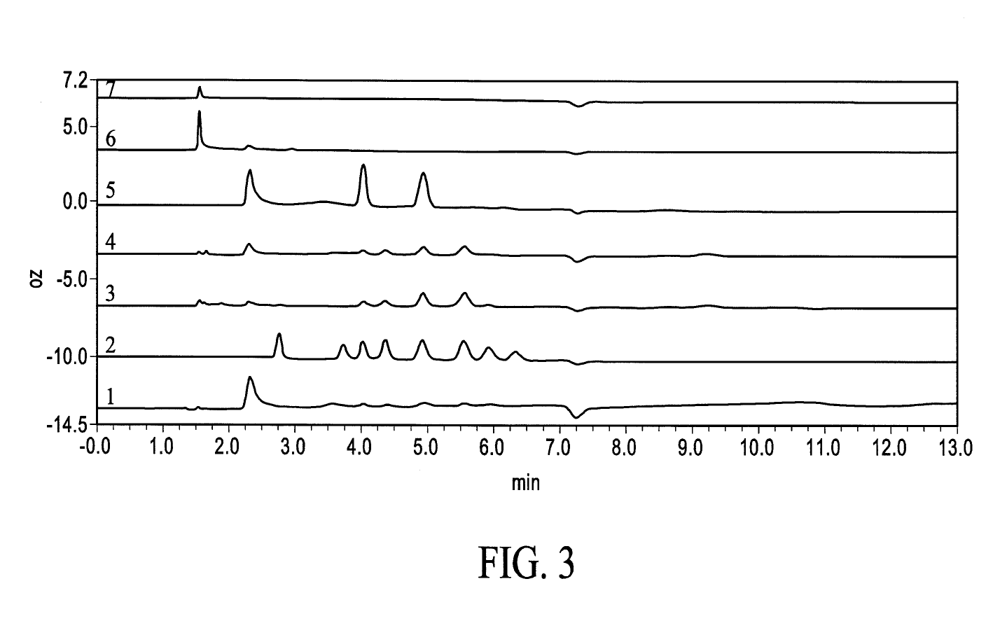

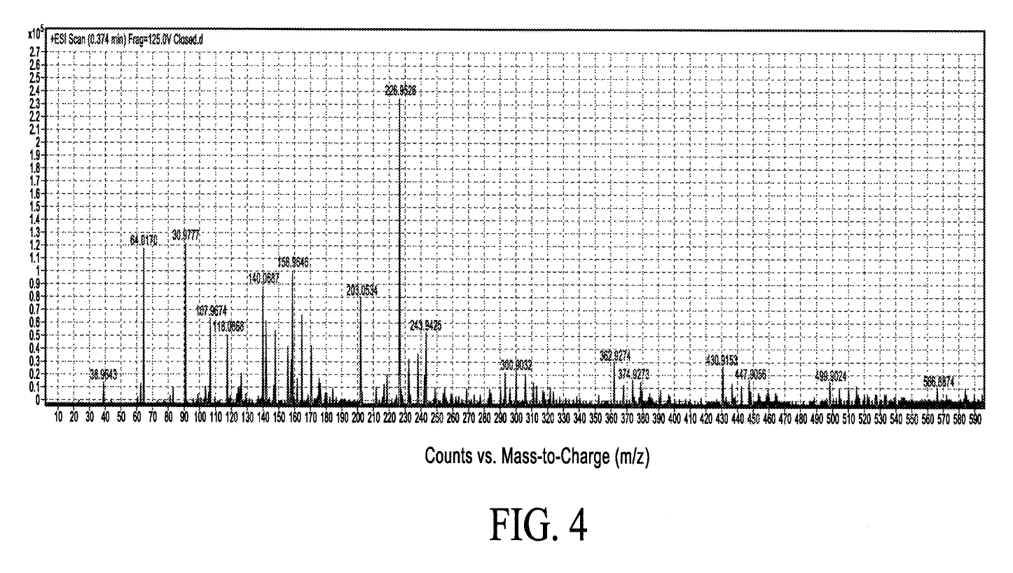

Furthermore, following biomass samples were treated with hydrogen peroxide and copper, at hydrogen peroxide concentrations of 3%, and the resulting samples were run on HLPC. FIG. 3 shows the results of the run. The fifth peak from the left is glucose. Samples from the top are, switch grass, corn stover, glucose control, sawdust, huge fuel, carbohydrates standards and paper. Furthermore, sawdust at 2.0 g was also treated with hydrogen peroxide at 35% and copper wire (0.5 g), at total volume of 50 ml, and the resulting liquid was run on MS. Results are shown in the FIG. 4. The main peak is glucose.

Level of degradation of glucose due to oxidation was determined by running the following samples: 30% H₂O₂ (negative control); 1% glucose solution in distilled water (positive control); 1% glucose in 30% H₂O₂; 1% glucose in 30% H₂O₂, and in the presence of 0.2 g copper foil; 1% glucose in 3% H₂O₂, and in the presence of 0.2 g copper foil.

Benedict's test was run on all samples. While the positive control and sample 3 showed positive results for the Benedict's test (presence of glucose), sample 4 was negative, even though there was glucose in the solution. Furthermore, sample 5 had some red precipitate; however, the level was not similar to that of positive control or sample 3. Results show that while glucose can be oxidized in the presence of hydrogen peroxide, it can be degraded in the presence of hydrogen peroxide and copper due to the catalytic activity of the copper. Furthermore, this degradation can be controlled either by the level of hydrogen peroxide or by the amount of catalyst.

Extraction of the glucose from the biomass, is accomplished as the result of the reaction of hydrogen peroxide with biomass in the presence of a metal cation. Either the concentration of hydrogen peroxide or the catalyst can be adjusted to obtain the high efficiency of the extracted glucose in the remaining solution (i.e. the level that is extracted and not degraded). To show that the extraction of glucose from biomass, can be accomplished using hydrogen peroxide and metal cations present in the biomass, metal content of the four biomass, used in industry (as described in the materials), were measured using an MS system. Table I shows the results of the run.

TABLE I
Metal content of the industrial biomass (ppb)

| | Fe | Cu | Ba | Ca | Mg |
|---|---|---|---|---|---|
| Switch grass | 480 | 92.5 | 1350 | 720 | 15000 |
| Sawdust | 6000 | 87 | 2800 | 1400 | 18000 |
| Hug fuel | 6400 | 100 | 5500 | 5400 | 45000 |
| Corn stover | 1200 | 100 | 650 | 1800 | 68000 |

Results shown in the Table I indicates that, the different types of biomass, used by the industry, contain metal cations, which can be used as catalysts for the hydrogen peroxide and metal catalyst system.

Furthermore, the onsite made biomass was treated with 30 ml 30% hydrogen peroxide, in the presence and absence of added copper as a catalyst. After the reaction was completed, the samples were filtered and the remaining liquid was dried and the residue was re-dissolved in 5 ml distilled water. The obtained samples were filtered again and read on a reafractometer and run on a Benedict's test. Table II shows the results.

TABLE II
Detection of glucose in the hydrogen peroxide treated biomass,
in the presence and absence of added copper catalyst

| Added copper | Refractometer reading | red precipitate |
|---|---|---|
| Yes | 2 | No |
| No | 9 | yes |

Results show that metal cations present in the biomass are enough to cause the extraction of glucose. The added copper does result in more extraction of glucose (as shown by the degree of degradation caused by the process), however, it also destroys the obtained glucose. Hence, the treatment of biomass with hydrogen peroxide or another oxidizing agent would result in a high yield of glucose due to the presence of metal cations in the biomass. This would not only reduce the cost of treatment, but also reduce or eliminate the waste due to the process.

### Solid Biomass

To eliminate solid biomass, there is a need for mechanical cutting of the materials, which is costly. Furthermore, to obtain energy from them, the treatment needed to accomplish this task is very harsh and costly. As has been demonstrated above, we can reduce the mass of the different types of biomass by the process of oxidation (using hydrogen peroxide and copper). To demonstrate the ability of this process to physically degrade a biomass material which is a hard solid, we processed some cardboard material. The hydrogen peroxide/copper treatment of the cardboard resulted in the complete degradation of the material.

Paper waste was treated with hydrogen peroxide (35%) and copper wire and completely reduced. Hence, this process can be used for waste treatment of “solid” biomass, not just water contaminants that are soluble or dispersed in water. Therefore, the cost of water decontamination can be much lower. As explained before, the level of degradation of the biomass can be controlled by the concentration of peroxide or the catalyst used. Therefore, as a part of solid biomass treatment, one can start by using a low concentration of peroxide and/or catalyst, and after obtaining the maximum level of glucose from the biomass one can increase the peroxide and/or catalyst concentration and cause the degradation of the remaining mass.

### Biomass as a Fuel to Generate Heat

Biomass is also used in industry as a fuel to generate heat. This is accomplished simply by burning the organic material of biomass. Generated heat can be used to boil water to get steam and subsequently generate electricity, or for some other purpose. As it is evident this procedure results in generation of CO₂ and the remaining burnt biomass is a waste. Furthermore, the biomass used for this procedure cannot be used for any additional purposes.

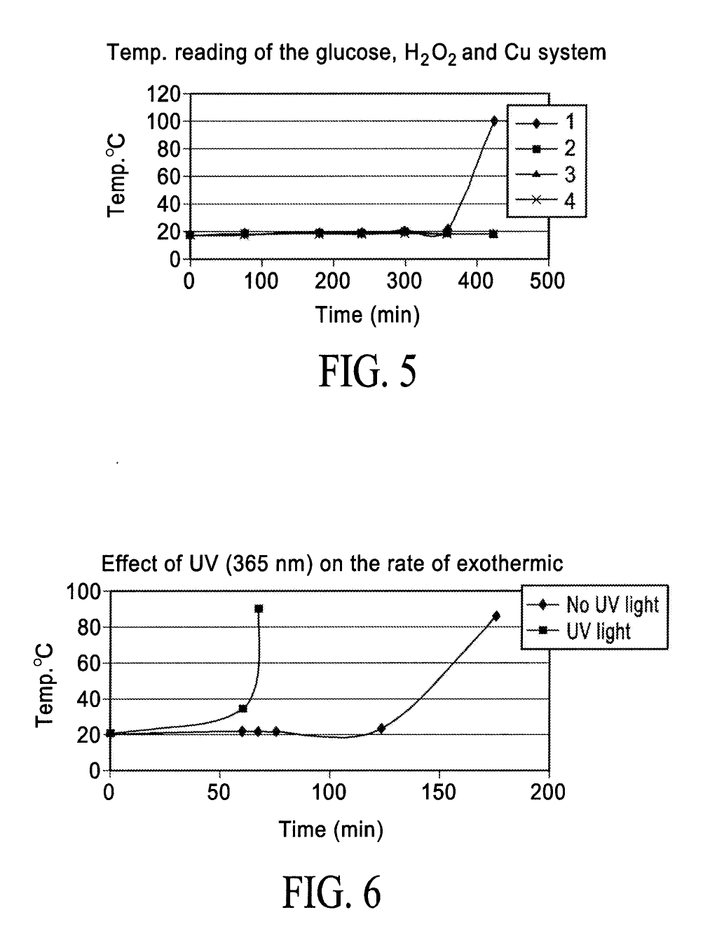

Oxidation of glucose is an exothermic reaction. However, the generated heat is not enough to be used for any practical purpose. We have observed that as the biomass is oxidized in the presence of hydrogen peroxide and copper, there is also heat generation. We postulated that the generated heat is due to the oxidation of methane and hydrogen gases generated during the oxidation of glucose content of the biomass during the process. To demonstrate this point, the following experiments were performed: glucose (5 g) was added to either hydrogen peroxide (30%) or distilled water, in the presence or absence of Cu wire, in an open container. Hydrogen peroxide with copper wire was used as control. Temperatures of all samples were monitored. FIG. 5 shows the results.

1. glucose+Cu+H2O2

2. glucose+Cu+distilled water

3. glucose+H2O2

4. H2O2+Cu

As shown in the FIG. 5, generated heat only occurred in the mixture of glucose, Cu and H₂O₂. Glucose and H₂O₂ in the absence of copper did not cause such a high temperature. Hence, even though addition of hydrogen peroxide to glucose can result in the oxidation of glucose, the generated heat is of no practical use. Furthermore, the rate of the exothermic reaction is enhanced by exposure of the mixture to UV light. As shown in the FIG. 6, mixture of glucose, hydrogen peroxide and copper, results in an exothermic reaction much faster in the presence of UV light. Effect of UV light exposure on generation of exothermic reaction was also demonstrated when hog fuel was used for biomass.

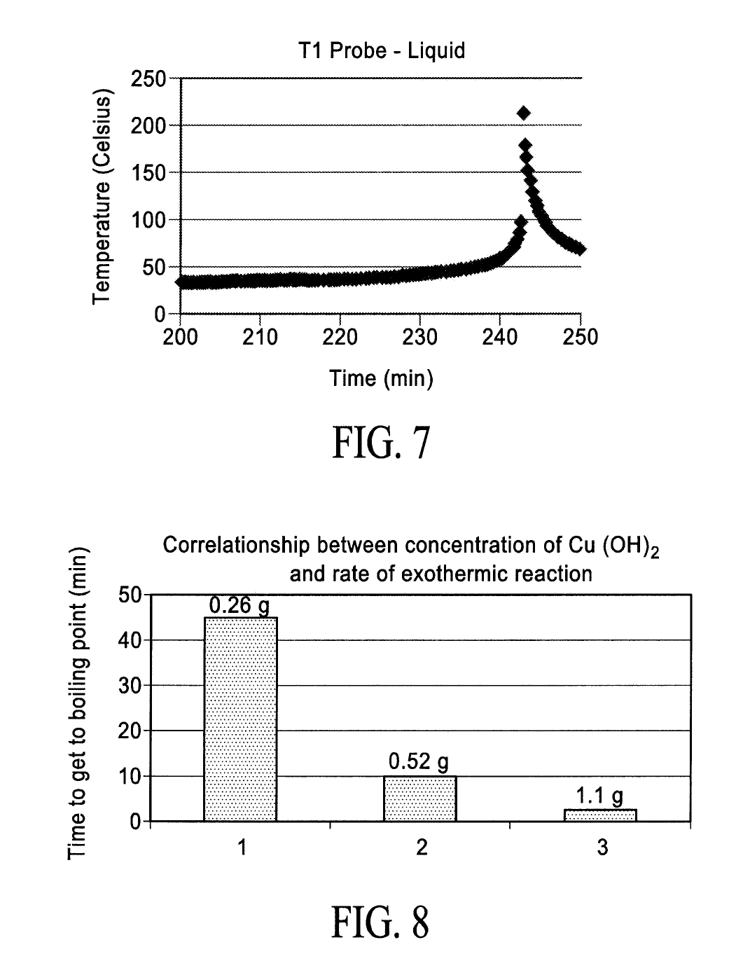

A reaction mixture of 0.6 g glucose, 0.1 g copper wire and 50 ml of 35% hydrogen peroxide was run in an incubator built for measurements of the heat and pressure generated during a reaction. Both the temperature and pressure generated during the reaction were measured. FIG. 7 shows the result of the measurement. The pressure generated in this reaction was 70 psi.

Since there is a blue precipitate forming during this process (either in the case when pure glucose is used or biomass is used), one theory is that the exothermic reaction happens, when Cu(OH)₂ is formed. To demonstrate this point, Cu(OH)₂ at increasing levels were added to hydrogen peroxide (30%), and the time needed to get to the boiling point was measured. Results are shown in FIG. 8.

As shown in FIG. 8, addition of Cu(OH)₂ to hydrogen peroxide alone could result in the high temperature, and the increase in the level of added Cu(OH)₂ to the solution resulted in the shorter time needed to get to the boiling point.

Therefore, by treating the biomass with copper, one not only can treat the biomass for ethanol generation, but also the treatment can be continued to generate heat from the same starting biomass, and finally the complete degradation and removal of the biomass. In addition, the generated heat and pressure can be used for other applications. The generated heat can be used to heat water and produce steam for electricity generation. Also, the generated pressure can be used to move a piston for a mechanical application, or for electricity generation.

Also, the heat and pressure generated by this method can be used to gasify biomass very inexpensively. Resulting gases can be used as a source of energy directly. Furthermore, as the graphs of the exothermic reactions show, the reaction happens in a spontaneous manner. Hence, it seems similar to an explosion. As stated before, one hypothesis is that the resulting methane and/or hydrogen gas(es) explode due to the oxidation and/or heat. This hypothesis is based on the fact that heat generated by oxidation of 0.6 grams of results in about 2200 calories. This is based on the reported value of 670 Kcal/mole of oxidized glucose. One calorie is the amount of heat needed to increase the temperature of one gram of water, one degree C. Hence, 2200 calories should increase the temperature of 50 ml solution by 44 C. Therefore, the temperature of the 50 ml reaction mixture in the FIG. 8 should not be more than 64 C (starting at RT, 20 C), whereas, the recorded temperature in the mixture is about 214 C. Required amount of calories for this increase is about 9700 calories. Also, increase in the temperature of the mixture is not due to the gas build up, resulting by the decomposition of hydrogen peroxide. The evidence for this is that the increase in the temperature of the mixture to the boiling point happens while the reaction is run in an open beaker. Hence, there are other chemicals with very high heat content produced during the reaction. Furthermore, since the temperature increase is higher in the closed container compared to that of open one, the generated materials are in the form of gas. Hence, methane and/or hydrogen are the possibilities.

We have demonstrated the use of hydrogen peroxide in the presence of a metal catalyst (such as Cu) to treat biomass, in an ethanol production procedure. We have further demonstrated that the same biomass can be used to generate heat and pressure in the same system, and finally degrade the starting biomass. Hence, this process allows one to treat a starting biomass material to not only obtain glucose (for ethanol production), but also obtain heat (an additional source of energy) and pressure. This process can be used to gasify biomass very cheaply, under ambient conditions. Also, continuation of the same treatment can result in the removal of the remaining waste.

While certain representative embodiments and details have been shown for purposes of illustrating the invention, it will be apparent to those skilled in the art that various changes in the methods and apparatus disclosed herein may be made without departing from the scope of the invention which is defined in the appended claims.

What is claimed is:

1. A method to degrade biomass comprising use of a peroxide in the presence of metal catalyst.

2. The method of claim 1 wherein the extraction of carbohydrate from biomass is controlled by the amount of metal cation catalyst used.

3. The method of claim 1 used as a cofactor to enhance the activity of enzymes used to digest biomass.

4. The method of claim 1 wherein the source of metal catalyst is either external or from a component of the biomass material.

5. The method of claim 1, wherein the peroxide is used at a concentration between 1% and 90%.

6. The method of claim 1 wherein the peroxide is either pre-made or manufactured onsite.

7. The method of claim 1, wherein a plurality of metal catalysts is used.

8. The method of claim 1, wherein the metal catalyst in the form of a free metal or a cation.

9. The method of claim 1, wherein the metal catalyst is in the form of bulk or nanoparticles.

10. The method of claim 1, wherein the system is used to generate heat.

11. The method of claim 9, wherein the metal catalyst is in a form selected from the group consisting of single metal cation and metal hydroxide.

12. The method of claim 11 wherein the plurality of metal cations and/or hydroxides is used.

13. The method of claim 1 used to generate pressure.

14. The method of claim 13 used for mechanical work.

15. The method of claim 13 is used to generate electricity.

16. The method of claim 1 used to gasify biomass.

17. The method of claim 16 used to generate fuel gases.

18. The method of claim 17 used to generate methane or hydrogen.

19. The method of claim 17, wherein the fuel gases are used directly as a source of energy.

20. The method of claim 17, wherein the fuel gases are used for applications, such as fuel cells.

21. The method of claim 1 used for the degradation and removal of solid biomass and/or organic waste.

22. The method of claim 1 used to extract chemicals and biochemicals from the biomass waste.

23. The method of claim 1 used in combination with UV light.

24. The method of claim 1 wherein sugar extracted is used for feed stock.

25. The method of claim 1 wherein the processed biomass is used for building materials.
